(19) United States
(12) Patent Application Publication
(10) Pub. No.: US 2025/0248497 A1
(43) Pub. Date: Aug. 7, 2025

(54) MEDICATION TRANSPORT WALLET AND METHODS OF USE

(71) Applicant: UCB BIOPHARMA SRL, BRUSSELS (BE)

(72) Inventor: FRANÇOIS BLIN, BRUSSELS (BE)

(21) Appl. No.: 18/429,507

(22) Filed: Feb. 1, 2024

(51) Int. Cl. A45C 11/24; A45C 11/00; A61M 15/00

(52) U.S. Cl. CPC A45C 11/24; A61M 15/00; A45C 2011/007; A61M 2209/06

(57) ABSTRACT

A medication transport wallet for discreetly carrying an inhaler is provided. The medication transport wallet includes front and back panels with distal ends secured with a tab arranged on an interior of the medication transport wallet. An aperture in the front panel allows access to a guide section of the tab that can be pushed toward the back panel when the inhaler is inserted into the medical transport wallet. The front and back panel compress against the tab and the inhaler to hold the inhaler in the medication transport wallet.

### BACKGROUND OF THE INVENTION

Several medical conditions can require urgent treatment in case of acute episodes. Many drugs are administered by means of an oral or nasal inhaler that persons can carry with them in the event of such an emergency situation. Examples for drugs that can be inhaled in acute medical conditions are anticholinergic and beta agonists drugs for the acute treatment of COPD; corticosteroids, anticholinergics, theophylline and beta agonists for treating asthma; analgesics such as fluranes or opioids for treating acute pain; insulin and derivatives for lowering blood sugar in diabetes; triptans, gepants, dihydroergotamine and prochlorperazine for treating episodes of migraine or cluster headache; neuroleptics for treating acute psychotic conditions; sedative/hypnotics for treating agitation, benzodiazepines for treating anxiety or panic attacks, anti-Parkinson drugs for treating off periods in Morbus Parkinson, and sedatives, antimigraine drugs and antiemetics for treating cyclic vomiting. Another condition that may require very quick action is epilepsy.

Epilepsy affects about 3.5 million people in the United States, with almost half a million of those afflicted being children. Epilepsy is a chronic disease and typically requires long-time treatment with anti-seizure drugs (anticonvulsants). Despite chronic treatment with these drugs, about 30-40% of the epilepsy patients are not seizure free but suffer at least from breakthrough seizures. While some of these patients may benefit from an optimized treatment regimen, e.g., by adding additional antiseizure drugs (polytherapy) or by trying alternative drugs, a substantial fraction of the patients remains not fully controlled and suffers from frequent seizures.

Epileptic seizures are the result of abnormal and uncontrolled electrical activity in the brain. They can occur rapidly and can render a person helpless. Most seizures stop spontaneously after a few seconds. However, some patients experience prolonged seizures that may require very quick pharmacological intervention to prevent the seizures from progressing to a more severe and potentially life-threatening stage. While rescue medications were traditionally administered at a rather late stage of significantly prolonged seizures such as e.g., after hospitalization, a much earlier pharmacological intervention is now recommended by most experts. A rapid early seizure termination (“REST”) has recently been advocated by patients and experts for a variety of reasons. The aim is to quickly intervene at an early seizure state to prevent the progression to a more severe seizure state and thereby (a) reduce the risk of hospitalization (b) prevent the transition to a life threatening, treatment refractory seizure state such as status epilepticus, and (c) reduce the risk of development into tonic-clonic seizures which is often accompanied with falls and injuries and also known to be associated with an increased risk of sudden unexpected death in epilepsy (“SUDEP”). Moreover, an effective REST medication allows the epilepsy patient to take better control over seizures and maintain independency.

The REST medication can be administered in the aura phase of the seizure or at a stage when the seizure does not self-terminate rapidly. In one instance, the REST medication can be administered if the patient has a history of prolonged seizure and/or a stereotypical development of seizures, which may lead the patient to self-administer the rescue medication at an early stage (e.g., in the aura phase) of the seizure. For example, certain forms of an aura or seizure development can indicate a predictable (stereotypical) evolution of such seizures such that early intervention is warranted. In some instances, the patient may decide to self-administer the REST medication in an early/aura phase of the seizure in order to immediately terminate the seizure in certain situations such as in the public or business environment, irrespective of seizure severity.

Drugs for the cessation of seizures (seizure rescue) can be any quickly acting antiseizure drug such as e.g., cannabidiol or benzodiazepines including, but not limited to, alprazolam, midazolam, diazepam, lorazepam, clonazepam, clobazam, bromazepam, flurazepam, oxazepam, estalozam, temazepam, triazolam, halazepam, premazepam, clorazepate, chlordiazepoxide or quazepam. Preferred drugs for the rescue therapy of epileptic seizures are benzodiazepines with established activity as antiseizure rescue medications such as midazolam, lorazepam, diazepam, clobazam, clonazepam and most recently, alprazolam. Midazolam and alprazolam are particularly preferred antiseizure rescue drugs in accordance with the present invention.

One example of a drug administered with an inhaler designed to be carried on a person for quick treatment of an oncoming seizure is disclosed in U.S. Pat. No. 11,241,383. This patent discloses a condensation inhaler that provides alprazolam in the form of a rapidly acting, orally inhalable aerosol. The device is breath-activated and can be used for self-administration but may also be used with the assistance of another person such as a caregiver. The inhaler can be disposable, i.e., for one time-use only. Alternatively, the inhaler can be designed for multiple use, where for example a part of the inhaler, including the drug and/or the heat producing entity of a condensation aerosol device, can be exchanged after use. In a preferred embodiment, the inhaler is disposable. The antiseizure inhaler can be used to stop or reduce the severity or duration of several forms of seizures. This includes but is not limited to seizures lasting longer than 2, 3, or 5 minutes such as prolonged focal seizures or prolonged generalized seizures including, but not limited to, those generalized seizures starting with a flurry of absence seizures or myoclonic seizures, seizures associated with juvenile myoclonic epilepsy, seizures wherein the patient has a history of seizures manifesting over a period of several minutes, or acute repetitive seizures (seizure clusters). The rescue medication may be administered in the early phase of a seizure, such as in the aura phase, in particular if the patient has a record of seizure clusters or prolonged seizures with a tendency to develop into generalized tonic-clonic seizures or even status epilepticus.

The drug-device combination is presently in advanced clinical trials and positive 2b data has been published recently (French et al., “A randomized Phase 2b Efficacy Study in Patients with Seizure Episodes with a Predictable Pattern Using Staccato® Alprazolam for Rapid Seizure Termination” Epilepsia, (2023), Vol. 64.3, pp. 374-385; French et al., “Two-part, Phase IIb Efficacy Study of Staccato® Alprazolam Inhaler in patients with Epilepsy with a Predictable Seizure Pattern: Topline Results from Part 2,” American Epilepsy Society 74th Annual Meeting—Virtual Meeting; Poster, Dec. 4-8, 2020).

The inhaler for the administration of the rescue medication such as alprazolam for REST treatment is preferably an inhaler to produce a condensation aerosol. The inhaler can comprise a first element for heating an alprazolam composition to form a vapor; a second element allowing the vapor to cool, thereby providing a condensation aerosol; and a third element permitting inhalation of the aerosol. Suitable heating elements are described in U.S. Pat. No. 11,241,383 and International Published Application No. WO2016/16145075 and involve a heatable/heat-conductive substrate which can be made of metal (e.g., steel or aluminum) and can be coated with a thin film of drug such as alprazolam. Typically, the substrate or support is heated to a temperature sufficient to vaporize all or a portion of the alprazolam film, typically at least 300° C., preferably at least about 350° C. or 390° C. +/−50° C., such that the composition forms a vapor rapidly, e.g., in less than 2 s, less than 1 s or in less than 0.5 s, that becomes entrained in a stream of air during inhalation. The second element that allows cooling is, in its simplest form, an inert passageway linking the heating element to the inhalation element. The third element permitting inhalation is an aerosol exit portal that defines a connection between the cooling element and the mammal's respiratory system, such as a mouthpiece. The alprazolam aerosol produced by such an inhaler has a mass median aerodynamic diameter (MMAD) of about 0.5 μm to 3.0 μm, which is optimal for deep lung delivery such that the pharmacokinetic of the administered alprazolam dose can be similar to an IV injection. Preferably, at least 80% or 90% by weight of the alprazolam aerosol particles have a size less than 5 microns and in one embodiment at least 50% by weight of the alprazolam aerosol particles have a size less than 2 microns. The heat for vaporizing the rescue drug such as alprazolam can be generated through an electrical resistance element; absorption of electromagnetic radiation, such as microwave or laser light; and chemically reactive material which undergoes an exothermic reaction upon actuation, e.g., by a spark or heat element. The rapid vaporization of the drug such as alprazolam leads to a very quick pulmonary administration and entry into the blood stream of the patient and a very quick onset of action with a medium time to seizure cessation of between about 30 and about 60 seconds, depending on the patient population and seizure type (French 2023).

To maintain the integrity of the drug and the potential heat source or other key elements in the device, inhalers, in particular disposable inhalers, are often vacuum sealed in a cover, which protects the integrity of the inhaler and the drug composition therein from environmental influences and the potential quicker aging such as drug decomposition by e.g., oxidation or inhaler dysfunction due to humidity. Protective covers for medication are frequently made of aluminum foil, due to the advantageous properties, above all its outstanding barrier properties where air and oxygen, light, humidity and/or water vapor and micro-organisms are concerned. Typically, the inhaler is carried with the patient and can be stored in handbags, backpacks and the like. However, the inventor of the present disclosure has identified that there is a risk that the outer cover will be damaged by other things carried together in the bag such as keys, smartphones, pens or other items risking the integrity of the protective atmosphere within the cover. This can lead to the inhaler losing function much earlier than the indicated half-life and can lead to less reliable pharmacological action if used in an emergency situation such as an epileptic seizure. In addition, covers made of aluminum foil are easily deformable, quickly leading to curled edges and corners that can prevent quick retrieval from storage through attachment to other objects.

For this reason, the present disclosure suggests that the inhaler and the outer cover be kept in an outer protective packaging or box to prevent the damage of the cover and protect the integrity of the inhaler. However, common packaging can be bulky, indiscreet, and inconvenient to carry on a person. Further, when the medical emergency episode, such as the seizure begins, very quick action may be required such that the patient has little time to prepare the inhaler prior to administration of the emergency drug. Unpackaging the inhaler can take time the patient or accompanying person may not have before the full medical emergency such as the seizure or other acute episode begins.

There is a need for patients suffering from a disease that includes acute severe episodes, particularly epileptic patients, to be able to safely carry an inhaler in a discreet manner that can also minimize the time necessary to prepare the inhaler for use. It would be convenient if such a carrying device could be included with the inhaler packaging.

### BRIEF SUMMARY

In accordance with embodiments of the subject invention, the problem of safely and discreetly carrying an inhaler sealed within a protective cover or pouch is solved with a transport wallet. The transport wallet can protect the sealed inhaler, such as an inhaler, while in the protective cover and prevent or reduce the damage of the protective cover and a potential early aging of the device components including the active ingredient. The transport wallet can also provide quicker access to said inhaler thereby reducing the time required to prepare the inhaler for pulmonary administration of the drug. Advantageously, the transport wallet can be less bulky than the original inhaler packaging, with front and back panels that are attached so they can flatten against each other when not in use, to be more convenient and less indiscreet when carried and can be conveniently packaged with the inhaler.

The travel wallet can be made of a variety of materials. In certain embodiments the travel wallet comprises an inexpensive paper or paper-like material, such as a stiff paperboard material (also sometimes referred to as cardboard). The paper or paper-like material can include, by way of example, wood, straw, hemp, cotton, bamboo, seed, grass or stone-based materials. In one particular embodiment, the paper is wood based. The wallet board material can also be decorated (printed) on all surfaces (recto and verso sides), with for example, the name of the medication contained therein. Alternatively, the exterior surfaces can be discrete without further imprint. In a particular embodiment, the wallet is configured for one-time use and can also be used to contain the depleted medication device after use.

In another aspect, the transport wallet of the subject invention comprises a front panel and a back panel that can be joined to form two substantially parallel lateral edges. The back panel can have a tab extending from the distal end with a section fold line that divides the tab into two sections: a fixation section and a guide section. The tab can bend or fold into an interior space between the back panel and the front panel. The fixation section, or a portion thereof, can be attached to the interior side of the front panel, such that at least a center area of the distal end of the front panel and the distal end of the back panel are held together or at least in close proximity. The guide section is unattached and capable of being bent at the section fold line toward the interior side of the back panel on the interior of the transport wallet.

The front panel can have an aperture near the distal end that is positioned over at least a portion of the guide section of the tab. Thus, at least a portion of the guide section can be seen and can be contacted through the aperture. Stated another way, the guide section covers at least a portion of the aperture, when the transport wallet is flat. Further, the aperture can be used to contact the tab to bend the tab at the crease and push, force, bend, urge, or otherwise move the guide section toward the back panel. The aperture can also be used to push the sealed inhaler towards the access at the proximal end.

The front panel and back panel can comprise a material having a bending stress that allows flexibility to the panels so they can bend and return to the same or approximately the same shape or condition. By squeezing or pressing together the lateral edges on each side of the transport wallet, an access can be opened between the front and back panels, which can bend outward, in opposite directions, at a proximal end of the transport wallet. The inhaler within the sealed protective pouch or cover can be slid into the access toward the distal end. The guide section of the tab can simultaneously be pushed or moved toward the back panel by contact through the aperture, so that the sealed inhaler slides between the tab and the front panel. When the inhaler is fully inserted the lateral edges can be released, which allows the front and back panels to unbend or unflex and come together to press against the inhaler therebetween. This can leave the access at least slightly open, which is efficacious for quickly removing the inhaler from the transport wallet. The additional compression at the distal end provided by the tab can increase resistance, stiction, or friction against the sealed inhaler to prevent or reduce the likelihood of accidental or unintended removal from the transport wallet, regardless of the orientation of the transport wallet.

A transport wallet of the subject invention can be formed or constructed from a flat one-dimensional sheet of material, which can be folded to form a 3-dimensional shape or carton, known in the art as a folding cartonboard. Folding cartonboard is often manufactured from various grades of paperboard materials, such as, for example, folding boxboard (FBB) or GZ or GN grades of Solid board. An embodiment of a folding cartonboard can be cut with a shape or outline resembling the profile of a medication transport wallet. The folding cartonboard can also have strategically placed fold lines that facilitate folding to form the three-dimensional medication transport wallet. In addition to the tab, discussed above, the back and/or front panel can have a flap extending from the lateral edge opposite the lateral edge shared between or common to both panels. When the folding cartonboard is folded, the shared lateral edge is formed where the front and back panels are folded along a score line. The other lateral edge can be formed when the flap is bent over and attached to the interior side of the opposite panel, to join the panels.

A transport wallet can be included as part of a kit within the outer packaging with an inhaler. When the outer packaging or box is opened, the inhaler, sealed within a pouch, wrapper, cover, bag, or other type of protective cover can be removed with the transport wallet. The inhaler in the protective cover can be inserted through the access into the transport wallet. The transport wallet can hold the packaged inhaler securely within and can also camouflage or at least partially conceal the protective cover for more discreet transport. The smaller profile transport wallet can also be more convenient to carry, protect the sealed cover of the inhaler from damage and the inhaler from potential dysfunction, and can reduce the time required to prepare the inhaler for use. After use, the inhaler can be returned to the transport wallet if desired, such as, for example, for disposal.

To use an inhaler, or other medical device or medication placed, contained, or otherwise, arranged in the transport wallet, the transport wallet can be held in one hand and the medical device or medication, contained within a protective packaging, sleeve, cover, or envelope, can be removed or slid out through the aperture with the other hand. The proximal edges around the access can also have an advantageous shape that facilitates at least partial visibility and grasping of the medical device or medication within the protective packaging, sleeve, cover, or envelope. This can further minimize the coordination required to utilize the medical device or medication. Once removed, the protective packaging, sleeve, cover, or envelope can be opened and the inhaler, or other medication, removed and administered as directed.

### BRIEF DESCRIPTION OF DRAWINGS

In order that a more precise understanding of the above recited invention can be obtained, a more particular description of the invention briefly/described above will be rendered by reference to specific embodiments thereof that are illustrated in the appended drawings. The drawings presented herein may not be drawn to scale and any reference to dimensions in the drawings or the following description are specific to the embodiments disclosed. Any variations of these dimensions that will allow the subject invention to function for its intended purpose are considered within the scope of the subject invention. Thus, understanding that these drawings depict only typical embodiments of the invention and are not therefore to be considered as limiting in scope, the invention will be described and explained with additional specificity and detail through use of the accompanying drawings in which:

FIG. 1 is a top, right side perspective view of an embodiment of a medication transport wallet, according to the subject invention.

FIG. 2 is a front-side elevation view of an embodiment of a medicine transport wallet, showing the aperture, according to the subject invention. The paired break lines indicate variable length.

FIG. 3A is a back-side elevation view of an embodiment of a medication transport wallet, according to the subject invention.

FIG. 3B illustrates alternative shapes for the proximal end of a panel.

FIG. 4 is a right-side elevation view of an embodiment of a medicine transport wallet, according to the subject invention.

FIG. 5 is a top-side plan view of an embodiment of a medicine transport wallet, according to the subject invention.

FIG. 6 is a bottom-side plan view of an embodiment of a medicine transport wallet, according to the subject invention. In this view the tab can be seen folded toward the interior.

FIG. 7 is front right-side perspective view, showing an embodiment of the medication transport wallet with an open access.

FIG. 8 is a front-side elevation view of an embodiment of the medication transport wallet with an open access. It can be seen how the lateral sides come together to separate the front and back panels to open the access.

FIG. 9 is a back-side elevation view of an embodiment of the medication transport wallet with an open access. It can be seen how the lateral sides come together to separate the front and back panels to open the access.

FIG. 10 is a right-side elevation view of an embodiment of the medication transport wallet with an open access. It can be seen how the front and back panels are separated to open the access.

FIG. 11 is a top-side plan view of an embodiment of the medication transport wallet with an open access.

FIG. 12 is a bottom-side plan view of an embodiment of the medication transport wallet with an open access. It can be seen in this figures that the distal end is held together or partially closed by the tab attached between the back panel and the front panel.

FIG. 13 is a cross-sectional view taken along line 13-13 in FIG. 8.

FIG. 14 shows an embodiment of one-dimensional folding cartonboard that can be folded into a three-dimensional medication transport wallet, according to the subject invention.

FIG. 15 shows an alternative embodiment of a one-dimensional folding cartonboard that can be folded into a three-dimensional medication transport wallet, according to the subject invention.

FIG. 16 shows an inhaler within a protective cover being inserted into an embodiment of a medication transport wallet, according to the subject invention.

FIG. 17 shows an embodiment of an inhaler kit that includes an outer package that contains an inhaler sealed within a protective cover (shown here being extracted from the protective cover) and a medication transport wallet, according to the subject invention.

### DETAILED DISCLOSURE

The subject invention pertains to devices and methods for carrying, storing, or transporting small, personal medical devices and methods of treating a condition utilizing the transported medical device. More specifically, the subject invention pertains to a transport wallet for containing, concealing, and protecting an inhaler, particularly a disposable inhaler to be carried on a person. Ready-for-use inhalers, in particular disposable inhalers, are typically sealed in a packaging, sleeve, cover, or envelope (herein referred to as a “cover”) to protect the medication therein from environmental influences but such a cover may not provide sufficient protection or privacy for the inhaler device. The inhaler in the sealed cover is usually further packaged in an outer-box, carton, or other protective outer container along with any other informational material or accessories. While helpful in protecting the inhaler in the commercial package, this outer packaging can be too bulky and inconvenient to discreetly carry on a person. The transport wallet of the subject invention advantageously allows the inhaler to be removed from the outer-container and discreetly carried in the sealed cover, while protecting the sealed cover of the inhaler from unintentional damage and the inhaler from accelerated aging or even from becoming dysfunctional. Furthermore, the transport wallet does not interfere with, and can facilitate, rapid use of the inhaler when necessary.

It should be understood that, while the following description is with respect to transporting and/or at least partially concealing or disguising an inhaler within a protective cover that contains a medication for treatment of a patient with a medical condition, this invention can also be used for transporting other items, such as medications that are administered without an inhaler or with other types of personalized medical devices, particularly those contained in a protective cover or the like, e.g., injector pens and blister packed medications. Consequently, while this invention can be particularly beneficial when carried on a person to provide immediate access to a medication, this does not preclude carrying or storing in another location or non-medical uses. In any event, this invention can be used whenever it is desired to safely and discreetly transport medication. It need not be limited solely to transporting an inhaler on a person.

In the description that follows, a number of terms are used in relation to the subject invention. In order to provide a clear and consistent understanding of the specification and claims, the following definitions are provided:

As used herein, the term “substantially” refers to the complete or nearly complete extent or degree of an action, characteristic, property, state, structure, item, or result. For example, an object that is “substantially” in a given position including but not limited to vertical, horizontal, or adjacent to or aligned with another object, would mean that the object is either completely in that position or nearly completely in that position. The exact allowable degree of deviation from absolute completeness can in some cases depend on the specific context. However, the nearness of completion will be so as to have the same overall result as if absolute and total completion were obtained.

Also as used herein, the terms “about” or “approximately” as applied to a numerical value or range of values are intended to mean that the recited values can vary within an acceptable degree of error for the quantity measured given the nature or precision of the measurements, such that the variation is considered in the art as equivalent to the recited values and provides the same function or result. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

Further, as used herein, and unless otherwise specifically stated, the terms “operable communication,” “operable connection,” “operably connected,” “cooperatively engaged” and grammatical variations thereof mean that the particular elements are connected in such a way that they cooperate to achieve their intended function or functions. The “connection” or “engagement” can be direct, or indirect, physical, or remote.

The transitional terms “comprising,” or “comprises” or grammatical variations thereof, which are synonymous with “including”, “includes”, “containing”, or “contains”, is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. Use of the term “comprising” contemplates other embodiments that “consist” or “consist essentially of” the recited component(s).

The terms “longitudinal distance” or “longitudinal length”, as used herein, refer to the longitudinal measurement or the distance extending along the long axis. For example, the longitude or longitudinal length of a tab is the distance or direction between the tab fold line at the proximal end and the distal end of the guide section.

Finally, reference is made throughout the application to the “proximal end” or “proximal direction” and “distal end” or “distal direction.” As used herein, the proximal end or proximal direction is that end typically oriented upwards when carried on a person. For example, an access can be at the proximal end of the transport wallet. Conversely, the distal end or distal direction of the device is that end typically oriented downwards when carried on a person. For example, the tab can be attached to the distal end of the back panel.

It is to be understood that the figures and descriptions of embodiments of the present invention have been simplified to illustrate elements that are relevant for a clear understanding of the invention, while eliminating, for purposes of clarity, other elements that may be well known. Those of ordinary skill in the art will recognize that other elements can be desirable and/or required in order to implement the present invention.

The present invention is more particularly described in the following examples that are intended to be illustrative only because numerous modifications and variations therein will be apparent to those skilled in the art. As used in the specification and in the claims, the singular for “a,” “an” and “the” include plural referents unless the context clearly dictates otherwise.

Any reference in this specification to “one embodiment,” “an embodiment,” “example embodiment,” “further embodiment,” “alternative embodiment,” “select embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

Anti COPD and anti-asthma drugs include beta agonists, corticoids, antihistamines and anticholinergic drugs. Examples of inhalation delivery of these drugs are widely known to a skilled person and a variety of inhalation medications for the treatment of asthma and COPD are commercially available on the market.

“Beta agonistic drugs” (short: beta agonists) suitable for the acute treatment of COPD or for treating asthma comprise non selective beta agonists such as propranolol, labetalol, nadolol or carvediol or more preferably beta2 adrenergic 2(sub)receptor-specific agonists; suitable beta 2 selective agonists include but are not limited to esmolol, bitolterol, fenoterol, hexoprenaline, isoprenaline, levosalbutanol, levalbuterol, orciprenaline, pirbuterol, Procaterol, albuterol (also known as salbutamol), terbutaline, reproterol, metaproterenol, arfometerol, bambuterol, clenbuterol, formoterol, salmeterol, carmoterol, indacaterol, milveterol, olodaterol, vilanterol. Preferred beta agonists for the purpose of the present disclosure are short acting beta-2 agonists (“SABAs”) such as esmolol, bitolterol, fenoterol, hexoprenaline, isoprenaline, levosalbutanol, levalbuterol orciprenaline, pirbuterol, Procaterol, albuterol (salbutamol), terbutaline, reproterol and in particular those with a very fast onset of action; those include fenoterol, reproterol, terbutaline, salbutamol and formoterol.

“Corticoids” suitable for treating asthma include but are not limited to glucocorticoids selected from amcinonide, alclometasone, beclometasone, betamethasone, clobetasone, clobetasol, eloprednol, clocortolon, dexamethasone, desoximetasone, diflucortolone, fluprednidene, fluocinolons, fluocortolons, fluticasone, flumethasons, halometasons, halcinonide, hydrocortisone, mometasone, triamcinolon, budesonide, methylprednisolone, prednicarbate, prednisone, prednisolone, rimexolone and esters thereof. Preferred glucocorticoids for the treatment of COPD and asthma are prednisone, dexamethasone, beclomethasone, fluticasone, budesonide, mometasone, ciclesonide. Flunisolide, and triamcinolone and esters thereof. Corticoids may also be administered in conjunction with other drugs for treating asthma such as beta agonists. For example, asthma inhalers may include combinations like fluticasone and salmeterol, budesonide and formoterol or mometasone and formoterol, as non-limiting examples.

“Anticholinergics” suitable for treating asthma or COPD include but are not limited to antimuscarinic drugs selected from ipratropium, tiotropium, oxitropium, aclidinium, glycopyrronium, glycopyrronium, umeclidinium and salts thereof. These drugs may be administered in combination with corticoids and/or beta agonists.

“Analgetics” include drugs effective for treating acute pain; preferred are those drugs with an effective dose in the low mg range and a quick onset of action such as opioids and certain fluranes. Non limiting examples of opioid-like analgetics for use as emergency treatments are morphine, fentanyl, hydromorphone, codeine, dihydrocodeine, hydrocodone, oxycodone, propoxyphene, dextropropoxyphene, methadone, meperidine, diphenoxylate, loperamide, pentazocine, butorphanol, nalbuphine, levorphanol, dipipanone, tramadol, etorphine, dihydroetorphine, buprenorphine, tapentadol, phetidine, sufentanil, alfentanil and in particular the quickly acting remifentanil. Non-limiting examples of non-opioid analgesic drugs are pentobarbital, thiopental, acetaminophene, cannabinoid, ibuprofen, lidocaine, ketamine, propofol, capsaicin and derivatives, ibuprofen, diclofenac, naproxen, meloxicam, ketorolac, parecoxib, metamizole, prochloperazine, chlorpromazine, metoclopramide, haloperidol, droperidol, gabapentin, pregabalin, orphenadrine, indomethacin, ketoprofen, celcoxib, rofecoxib, meclofenamic acid, fenoprofen, diflunisal, tolfenamic acid, flurbiprofen, nabumeton and fluranes such as methoxyflurane, isoflurane, sevoflurane, enflurane or desflurane. Preferred examples of inhalative non-opioid drug for treating acute pain are cannabinoid and certain fluranes such as methoxyflurane.

“Insulin and derivatives” include insulin and active fragments and variants thereof, whether purified from natural sources or based on recombinant or other synthetic processes, which are effective in lowering blood sugar in diabetes; preferred examples are quick acting derivatives such as insulin aspart, insulin glulisine or insulin lispro.

“Anti-migraine drugs” include triptans, gepants, lisuride, dihydroergotamine, lidocaine, verapamil, diltiazem, and prochlorperazine. “Triptans” include sumatriptan, frovatriptan, naratriptan, rizatriptan, eletriptan, donitriptan, almotriptan, avitriptan and zolmitriptan. “Gepants” are calcitonine-gene related peptide (CGRP)-receptor antagonist which include ubrogepant, atogepant, rimegepant and zavegepant. “Anti-migraine drugs” might be also used for treating cluster headache, in particular triptans such as sumatriptan.

“Neuroleptics” for treating acute psychotic conditions include olanzapine, aripiprazole, ziprasidone, haloperidol, risperidone, asenapine, loxapine, quetiapine, trifluoperazine, clozapine, promazine, thiothixene, chlorpromazine, droperidol, prochlorperazine, and fluphenazine. Preferred examples are the quick acting loxapine, olanzepine and haloperidol.

“Sedatives/hypnotics” include barbiturates, benzodiazepines and non-benzodiazepine drugs. “Benzodiazepines” include alprazolam, midazolam, diazepam, lorazepam, clonazepam, clobazam, bromazepam, flurazepam, oxazepam, estalozam, temazepam, triazolam, halazepam, premazepam, clorazepate, chlordiazepoxide or quazepam.

“Barbiturates” include quickly acting drugs such as thiopentone (thiopental), methohexitone, hexobarbitone, cyclobarbitone, pentobarbitone and secobarbitone.

“Non-benzodiazepine sedatives” include zaleplon, zolpidem, zopiclone and eszopiclone.

“Antihistamines” include azatadine, azelastine, mizolastin, olaptadine, rupatadine, bilastine, brompheniramine, carbinoxamine, diphenhydramine, chlorpheniramine, dexchlorpheniramine, terfenadine, tripolidine, clemastine, cyproheptadine, loratadine, desloratadine, cetitizine, levocetirizine, acrivastine, fexofenadine, pyrilamine, doxylamine, phenindamine, dimenhydrinate, hydroxyzine, and promethazine. Preferred antihistamines are quickly acting 2^{nd} or 3^{rd} generation antihistamines such as cetirizine, levocetirizine, loratadine, mizolastine, azelastine or acrivastine.

“Anti-Parkinson drugs” include levodopa, apomorphine and dopamine agonists such as rotigotine, pramipexole, piribedil or ropinirole. A preferred drug for treating off periods in Morbus Parkinson is apomorphine.

“Antiemetics” include serotonin, neurokinin1 and/or dopamine receptor modulators such as aprepitant, amisulpride, alizapride, domperidon, droperidol, palonosetron, ondansetron, dolasetron, tropisetron, and in particular granisetron.

Reference will be made to the attached figures on which the same reference numerals are used throughout to indicate the same or similar components. With reference to the attached figures, which show certain embodiments, it can be seen that the subject invention comprises a transport wallet 100 that can have front panel 105 and a back panel 110 that define an interior space 120 for receiving an inhaler 16. In one embodiment, the front and back panels can be attached by a tab 150. The tab can extend from a distal end 10 of the back panel and fold into the interior space. A section fold line 156 on the tab can define a fixation section 158 and a guide section 160 on the tab, where the fixation section can be attached to the front panel on the interior side 116. The guide section can bend at a section fold line 156. In a further embodiment, there can bean aperture 106 through the front panel 105 that can be utilized to urge, push, move, or otherwise contact the guide section so the tab bends or folds at the section fold line toward the back panel in the interior space. The aperture can also be utilized to push or urge the sealed inhaler towards the access for removal. Each of these general components can have one or more sub- components, which will be discussed in detail below.

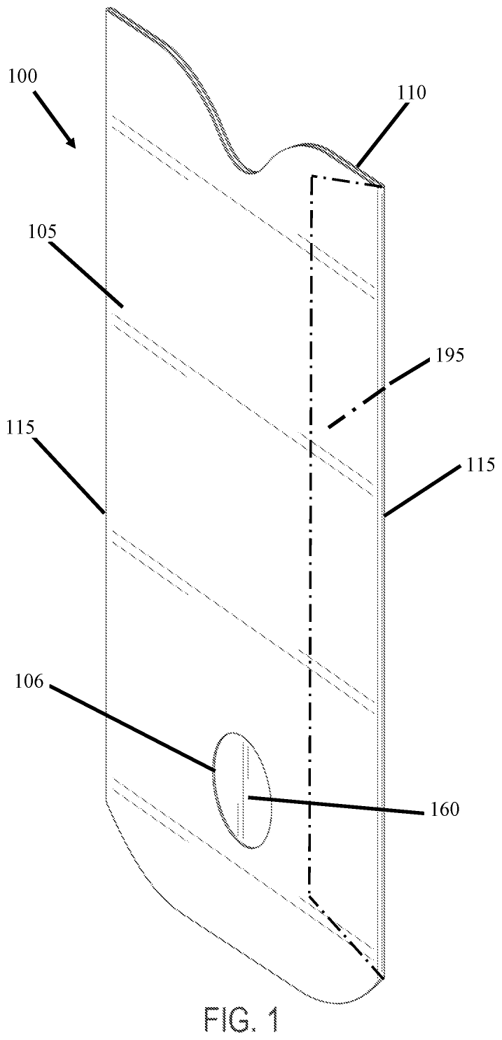

The structure of a transport wallet 100 of the subject invention can be such that there is sufficient resiliency, flexibility, or material memory to squeeze or press the sides or lateral edges 115 causing the front panel 105 and back panel 110 to bend outwards in opposite directions. This bending can create an opening or access 118 at the proximal end 5 for receiving the sealed inhaler 16. When the lateral edges are released, the front and back panels at least partially unbend or unflex, so they at least partially flatten toward each other and their interior sides 116 press against the sealed inhaler therebetween. The resiliency or flexibility required for bending the front and back panels can be dependent on selection of one or more manufacture materials with a desirable durometer. Ideally, the dimensions of a transport wallet with an inhaler therein allows for carrying on a person, such as, for example, in a pocket. It can be further desirable for a transport wallet to be manufactured from one or more lightweight materials with a desirable durometer and strength to provide a transport wallet with a generally thin, discreet, profile, such as shown, for example, in FIGS. 1 and 4.

A “folding cartonboard” refers to a sheet of material, usually made of a flat paperboard material, which can be folded to form a 3-dimensional container. There are a variety of paperboard materials, known in the art by different “grades” that can be utilized for folding cartons. Folding Box Board (FBB) is one type of material that can be utilized and has two grades: GC1 and GC2. Another type of material that can be used is Solid Board, which also has two grades: GZ (or SBS) and GN (or SUB).

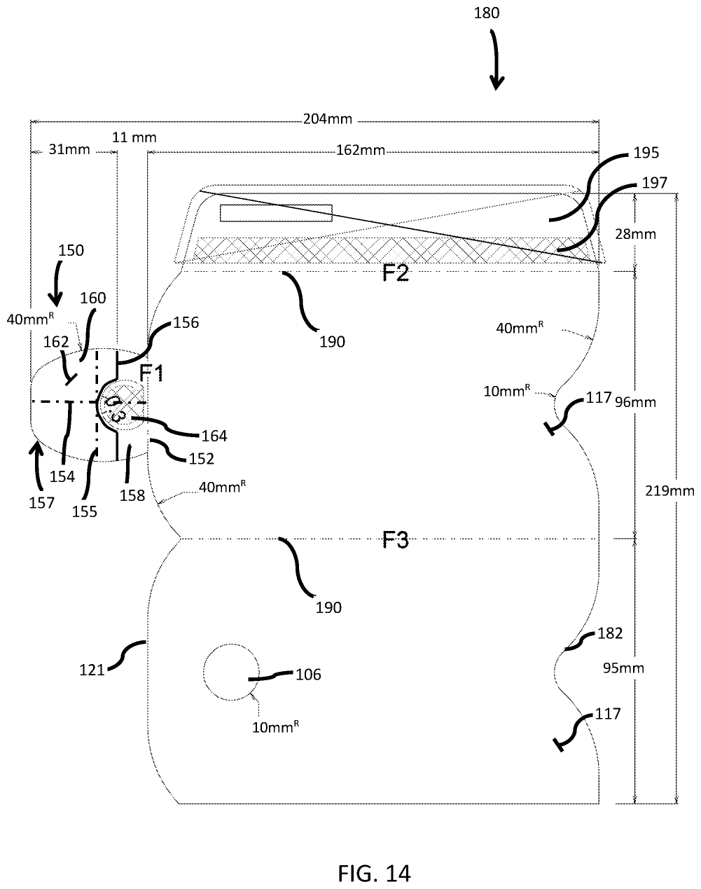

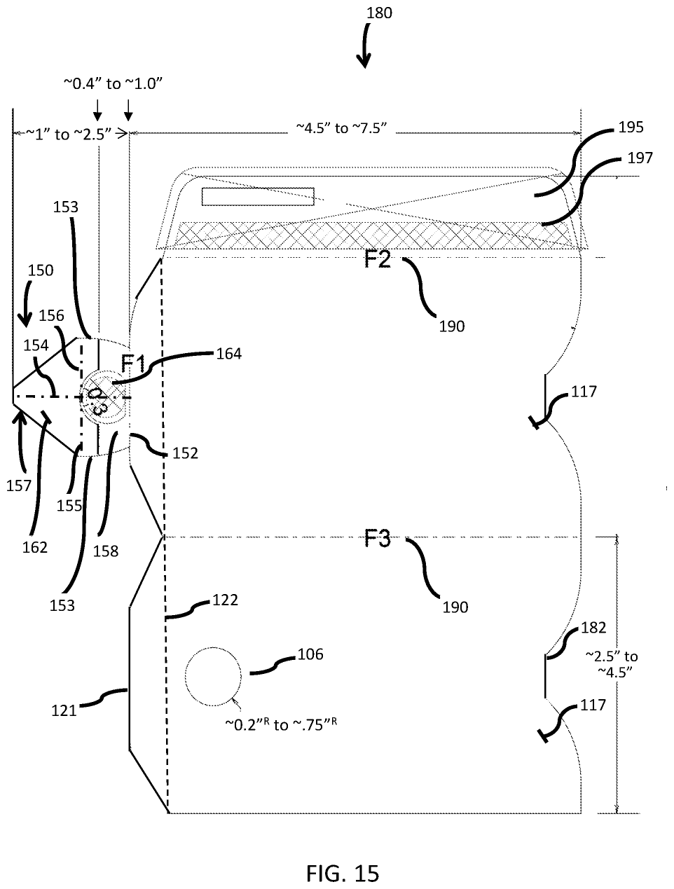

A transport wallet of the subject invention can also be constructed or assembled from a flat or one-dimensional sheet of material or “folding cartonboard” 180 that can be folded to form the 3-dimensional transport wallet. The cartonboard utilized for a folding cartonboard that can form a transport wallet can comprise any of a variety of paperboard materials that can be cut, such as, for example, by die cutting, to have a circumference or shape 182 resembling the outline or profile of a medication transport wallet 100. In one embodiment, the transport wallet can be formed from a folding cartonboard 180 manufactured from a Folding Boxboard (FBB) material. The folding cartonboard can also have one or more strategically placed fold or score lines 190. When folded along the pre-defined fold or score lines, the three-dimensional transport wallet can be formed from the one-dimensional folding cartonboard. One or more flaps 195 on the folding cartonboard can be used after folding to secure the three-dimensional shape of the transport wallet. FIGS. 14 and 15 illustrate embodiments of folding cartonboard 180 formed from folding boxboard (FBB) material that can be folded into a transport wallet 100. The folding cartonboard 180 will be discussed in more detail below.

In one embodiment, a transport wallet is manufactured from FBB or other stiff paperboard material. In a further embodiment, the stiff paperboard or FBB material has a food grade coating that provides a smooth outer surface to the transport wallet and is amenable to a variety of printing procedures. In one embodiment, the material has a grammage of between approximately 255 g/m² and approximately 280 g/m² and a thickness of between approximately 400 μm and approximately 450 μm. In a particular embodiment, the material has a grammage of approximately 270 g/m² and a thickness of approximately 425 μm. In a further embodiment, the material has a bending stiffness of between approximately 8.1 CD mNm and approximately 10.7 CD mNM and between approximately 16.2 MD mNm and approximately 21.4 MD mNm. In a still more particular embodiment, the stiff paperboard or FBB material has a bending stiffness of approximately 9.7 CD mNm and approximately 19.4 MD mNm. In a specific embodiment, the material of a transport wallet is considered a GZ or GC1 grade coated folding boxboard (FBB) in compliance with DIN ISO 19303. In more specific embodiment, a transport wallet is formed from a folding cartonboard 180 manufactured from GZ or GC1 grade FBB in compliance with DIN ISO19303.

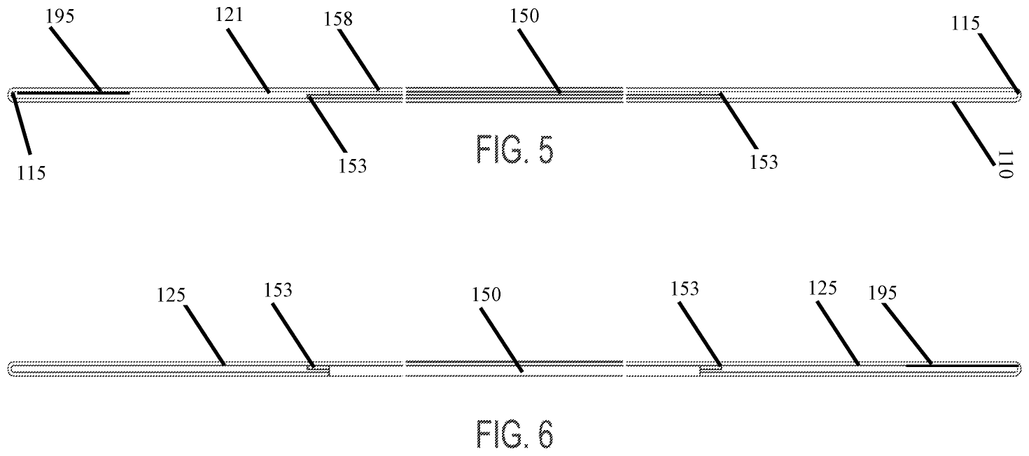

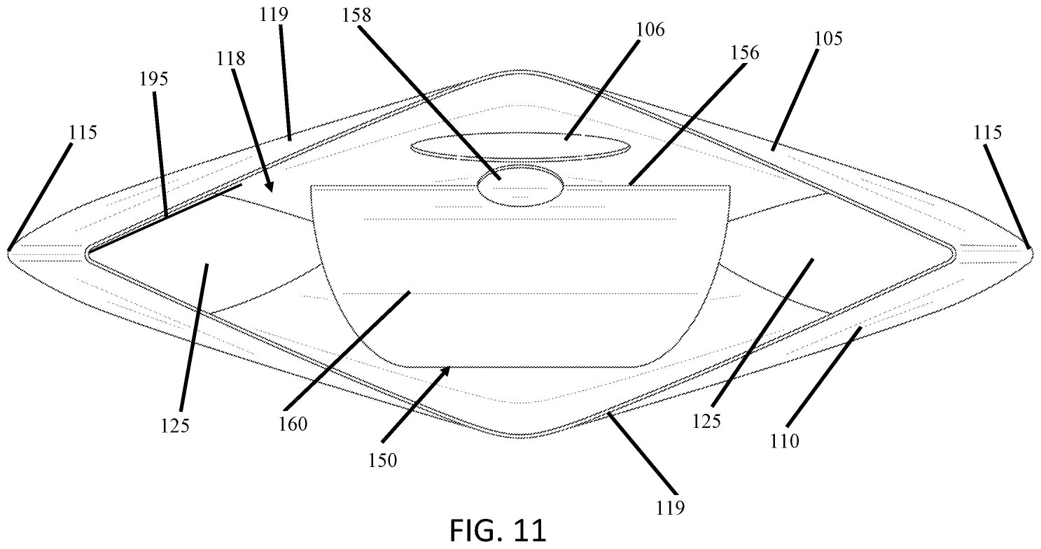

A medication transport wallet 100 of the subject invention can have a front panel 105 and a back panel 110 that are flat, each having an interior side 116 and an exterior side 117. The panels can have interior sides that face each other and that are connected at two substantially parallel sides or lateral edges 115, where the panels are joined or come together to form an interior space 120 for receiving the inhaler, which is shown, by way of example, in FIGS. 5 and 11.

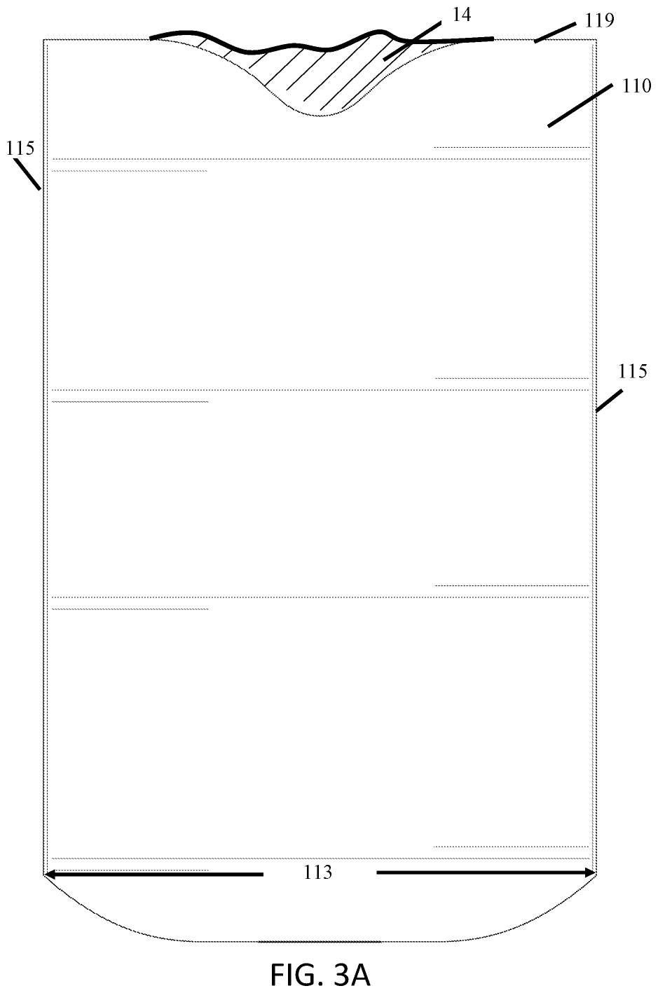

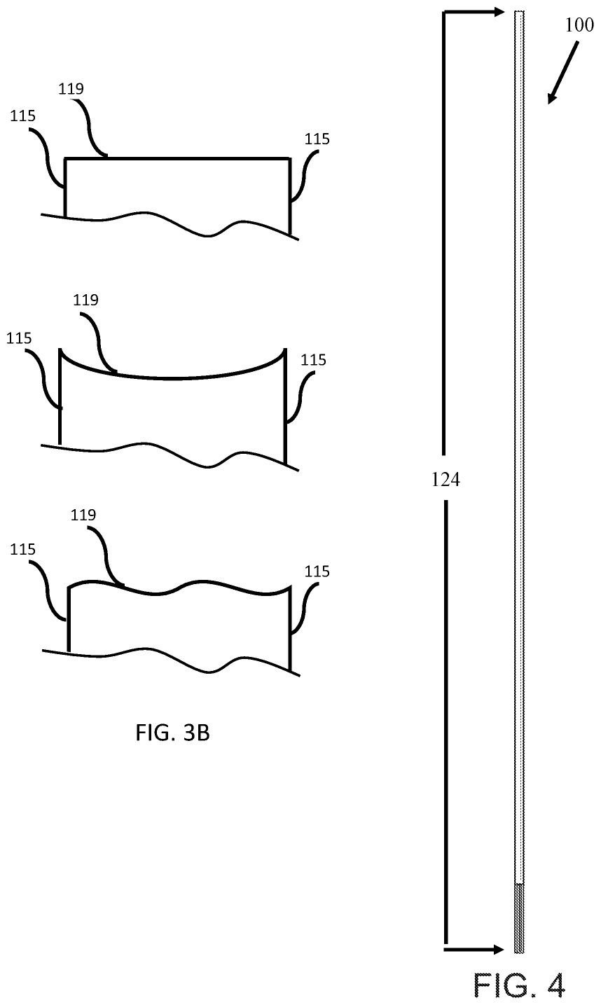

The front panel 105 and back panel 110 can have proximal edges 119 with the same or substantially the same outline or shape, so as to be congruent. Alternatively, the proximal edges can be mirror images, such that when viewing the exterior side 117 of a panel the proximal edge 119 is parallel with the proximal edge of the panel behind, such that the panel behind can be hidden by the panel in the front. FIG. 3A illustrates proximal edges with the same curved shape on the front and back panel, such that when viewed from the exterior side of one panel, the opposite panel is not seen or is not easily seen. FIG. 3B illustrates alternative configurations of proximal edges.

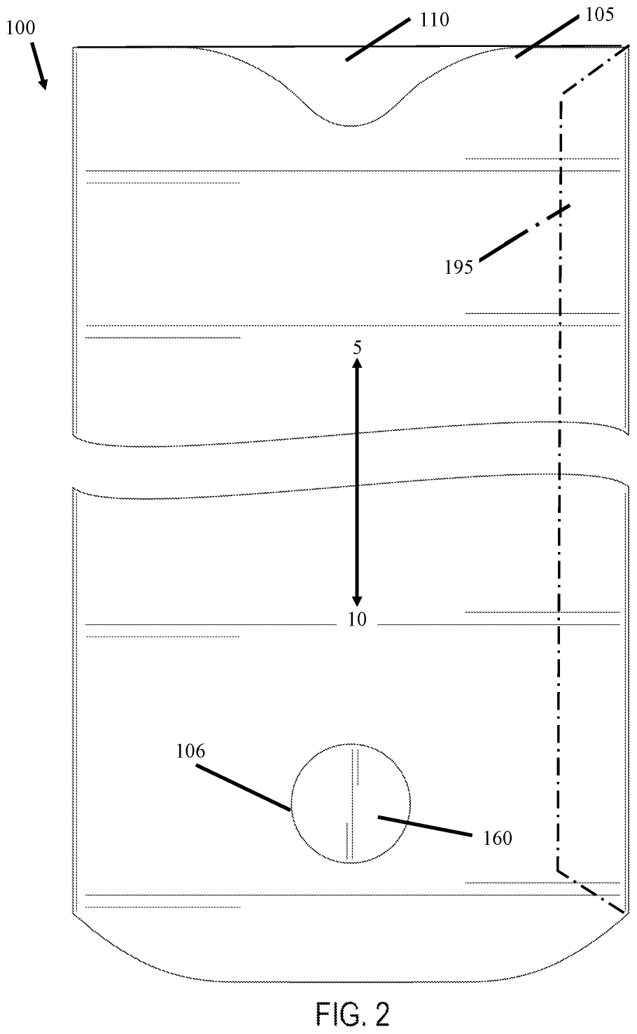

Alternatively, the front and back panels can have proximal edges 119 with different outlines or that are not mirror images, as shown, for example, in FIG. 2. In another alternative, the panels can have the same outline or shape, but are not parallel. For example, the proximal edge of one panel is higher than the proximal edge of the other panel. When viewed from the exterior side of one panel, the proximal edge or some portion thereof of the opposite panel can be seen. FIG. 2 illustrates an example of panels with proximal edges that are not parallel or that are not congruent or are not mirror images. In one embodiment, the proximal edge of one or both panels has a shape that is efficacious for removing the inhaler 16 within a protective cover 14 from the transport wallet 100. In a more specific embodiment, a portion of the proximal edge is lower or more distal than another portion, such that at least a part of the protective cover can be seen and grasped for removal from between the panels. A non-limiting example of this is shown in FIG. 3A.

The front panel 105 and the back panel 110 can also have distal edges 121. The distal edges can be secured to close, or at least partially close, the distal end 10 of the transport wallet. For example, the distal edges, or a portion thereof, can be glued, taped, stapled, crimped, heat sealed, sewn, or otherwise joined together by devices and techniques known in the art.

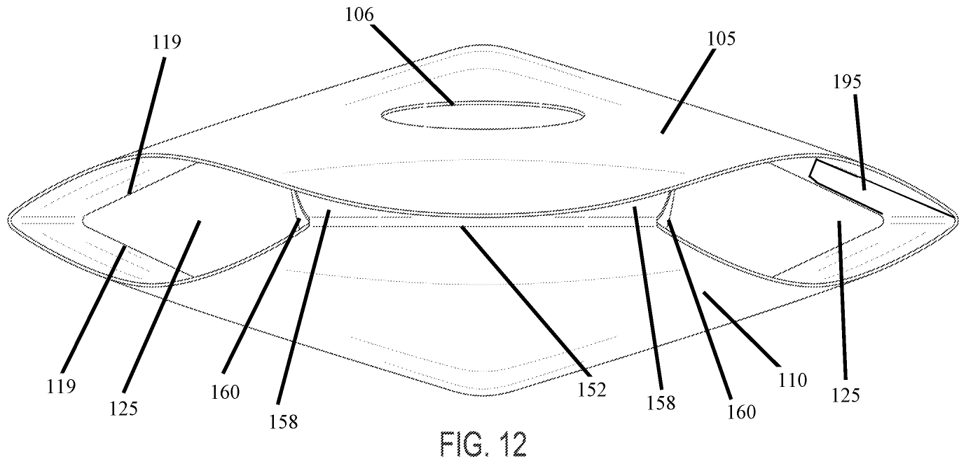

Alternatively, the back panel can have a tab 150 extending from the distal edge 121. By way of non-limiting example, a tab can extend from the center of the distal edge of the back panel, as shown, for example, in FIGS. 12, 13, 14, and 15. The tab can have a section fold line 156 that divides the tab, as shown in FIGS. 14 and 15, into two sections: a fixation section 158 and a guide section 160. There can further be a tab fold line 152 between the fixation area and the distal end of the back panel at which the tab can bend or fold into the interior space 120 of the transport wallet, between the back panel 110 and the front panel 105.

The tab can have a longitudinal length 154, which extends from the proximal end 5 where it attaches to the distal end 10 of the back panel to the furthest end from the distal end, and a width 155, which extends between the tab sides 153, which are shown in FIGS. 14 and 15. The perimeter or tab profile 157 can have any of a variety of shapes, including those with straight lines, curves, or curvilinear combinations thereof. FIGS. 14 and 15 illustrate non-limiting examples of tab profiles that can be utilized with embodiments of the subject invention. As will be discussed in more detail, the tab can provide additional compression on the packaged inhaler 16 to prevent or reduce the likelihood of the inhaler from accidentally or unintentionally falling out of the transport wallet. Thus, it can be beneficial for the guide section 160 to have sufficient surface area so that when compressed or pushed against the packaged inhaler by the back panel, there is a frictional resistance between the guide section 160 and the protective cover 14 of the inhaler to assist the interior sides 116 of the front and back panels in holding the packaged inhaler within the transport wallet 100 interior space 120.

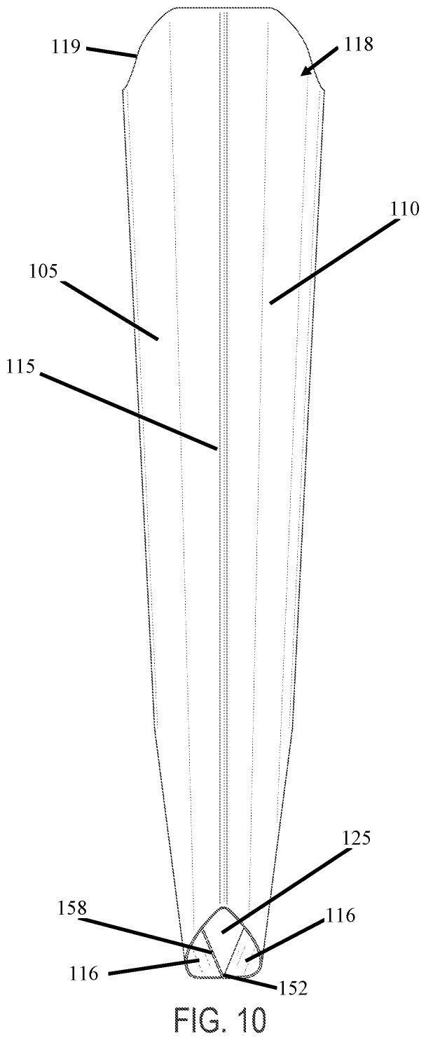

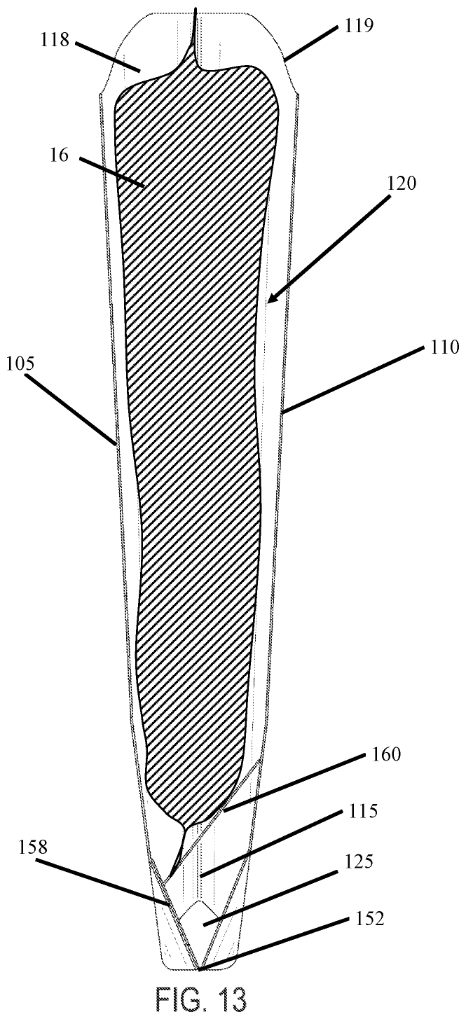

When the tab 150 is folded into the interior space 120 of the transport wallet 100, a back side 162 of the tab is disposed toward the front panel interior side 116, which can be seen, for example, in FIGS. 12, 13, 14, and 15. This allows the back side 162 of the fixation section 158, or a portion thereof, to be attached to the front panel interior side 116. More particularly, the fixation section or a portion thereof can be attached so that the distal end 10 of the front panel 105 is positioned or fixed at or near the distal end of the back panel 110, so that they are held together at least where the panels are connected with the tab. By way of a non-limiting example, as shown in FIGS. 14 and 15, the fixation section can have a fixation area 164 on the back side 162 of the fixation section 158. The fixation area can be attached to the interior side 116 of the front panel 105 by any of a variety of devices, materials, and techniques known to those with skill in the art, including, but not limited to, adhesives, adhesive strips, hook-and-loop material, crimping, heat-sealing, and snaps. The guide section 160, proximal 5 to the fixation section, can remain unattached and capable of bending along the section fold line 156, an example of which is shown in FIGS. 10 and 13.

The longitudinal length 154 and the width 155 of a tab 150 can each independently vary based on factors that include, but are not limited to, the material of the tab, the material of the protective cover 14 enclosing the inhaler 16, the dimensions of the sealed inhaler, the size of the fixation area 164, the width of the distal end 10 of the front 105 and back 110 panels and other factors known to those with skill in the art. The tab can also close or block at least a portion of the distal end 10 of the travel wallet, such that there can be one or more slits 125 between the front and back panels at the distal end that open to the interior space 120. FIGS. 5, 6, 11, and 12 show non-limiting examples of slits at the distal end of a travel wallet.

In a particular embodiment, the tab closes or blocks a portion of the distal end that is at or near the center of the travel wallet, such that one or both sides of the distal end to the sides of the tab are open to the interior space 120. Thus, a tab can enclose a portion of a distal end width 122 of the transport wallet. Embodiments of a tab 150 can have a fixation section that blocks or covers the distal end width of the transport wallet by at least about 10%, 15%, 20%, 25%, 30%, 35%, 40%, 45%, 50%, 55%, 60%, 65%, 70%, 75% and/or a percentage of the distal end width of a travel wallet that is between any two of the listed values. In a specific embodiment, the width 155 of a tab 150 is not equivalent to the width of the tab fold line 152. In a further embodiment, a tab can have a longitudinal length 154 of at least about 1.0″, 1.1″, 1.2″, 1.3″, 1.4″, 1.5″. 1.6″, 1.7″, 1.8″, 1.9″, 2.0″, 2.1″, 2.2″, 2.3″, 2.4″, 2.5″, and/or a longitudinal length between any two of the listed values. In an alternative embodiment, a tab can have a longitudinal length 154 of at least about 25 mm, 28 mm, 31 mm, 34 mm, 37 mm, 40 mm, 43 mm, 46 mm, 49 mm, 52 mm, 55 mm, 58 mm, 61 mm, 64 mm, and/or a longitudinal length 154 between any two of the listed values. In a still further embodiment, the longitudinal length of the tab includes the length of the fixation section 158, which can be at least about 0.4″, 0.5″, 0.6″, 0.7″, 0.8″, 0.9″, 1.0″, and/or a length between any two of the listed values. In an alternative embodiment, the longitudinal length of the tab includes the length of the fixation section, which can be at least about 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, and/or a longitudinal length between any two of the listed values. In a specific embodiment, the length of the fixation section is approximately 25% of the total longitudinal length of the tab.

The front panel 105 can have a hole or aperture 106 near the distal end 10, but proximal 5 to where the back side 162 of the fixation section 158 is attached to the front panel 105. In one embodiment, the hole or aperture can be proximal to the fixation area 164 that attaches to the interior side 116 of the front panel. More specifically, the aperture can be positioned over at least a portion of the guide section 160 of the tab 150 on the interior space 120 of the transport wallet. Thus, the aperture can be utilized to contact the tab, more specifically to contact the guide section 160 of the tab. The aperture can be appropriately shaped and sized to permit contact therethrough to bend the tab at the section fold line 156 so the guide section moves toward the back panel. The aperture can also be used to, at least partially, push the sealed inhaler out of the transport wallet. In one embodiment, the aperture has a maximum diameter of at least about 0.4″, 0.5″, 0.6″, 0.7″, 0.8″, 0.9″, 1.0″, 1.1″, 1.2″, 1.3″, 1.4″, 1.5″ and/or a maximum diameter between any two of the listed values. In an alternative embodiment, the aperture has a maximum diameter of at least about 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, and/or a maximum diameter between any two of the listed values.

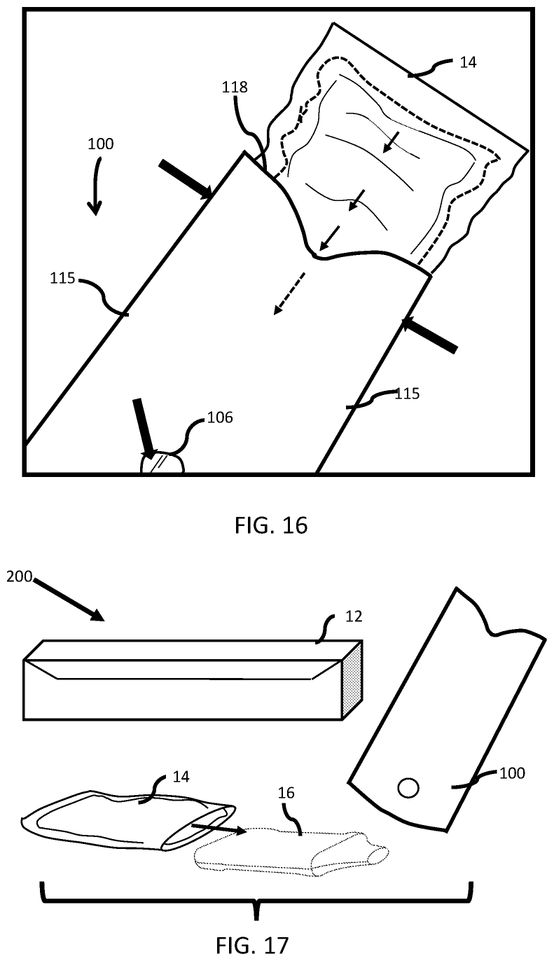

By squeezing the lateral edges 115 of the front 105 and back 110 panels together, an access 118 can be opened at the proximal end 5 of the transport wallet 100 when the panels bend or bow outwards, which can be seen, for example, in FIGS. 10 and 11. An inhaler such as a disposable inhaler within the sealed protective cover 14 can be slid into the access toward the distal end 10 of the transport wallet, which is shown, for example, in FIG. 16. The tab 150 can be simultaneously pushed or otherwise moved toward the back panel through the aperture 106, so that the inhaler in the protective cover can be inserted between the tab and the front panel. In a specific embodiment, the protective cover 14 is a vacuum pouch with one or more sealed edges. In a further specific embodiment, the protective cover 14 is an aluminum foil vacuum pouch. When inserted into the transport wallet, the sealed edge can insert between the tab and the front panel interior side. When the inhaler in the protective vacuum pouch is fully inserted into the travel wallet 100 the lateral edges can be released, which allows the front and back panels to at least partially unbend or come closer together to press against the inhaler therebetween. This can also allow the access to remain at least slightly open, which can be efficacious for quickly removing the sealed inhaler from the transport wallet.

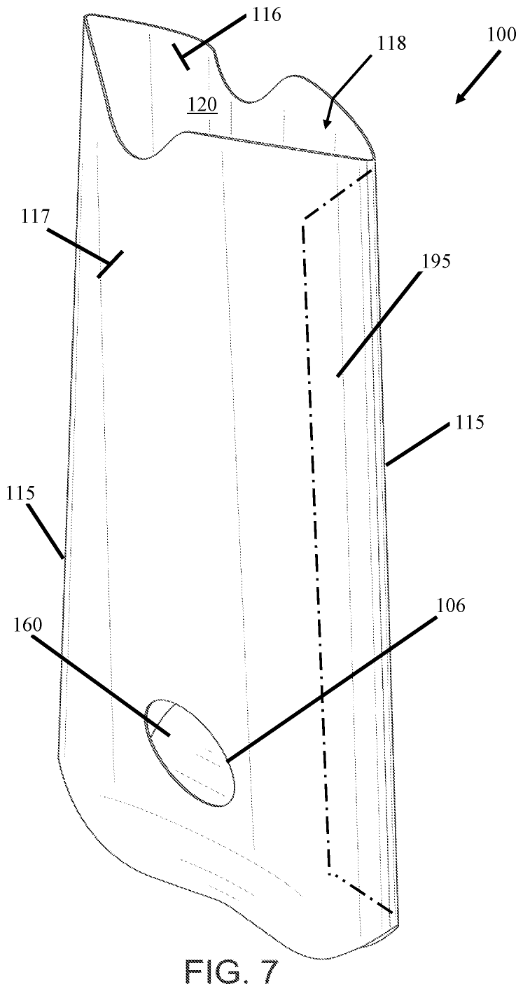

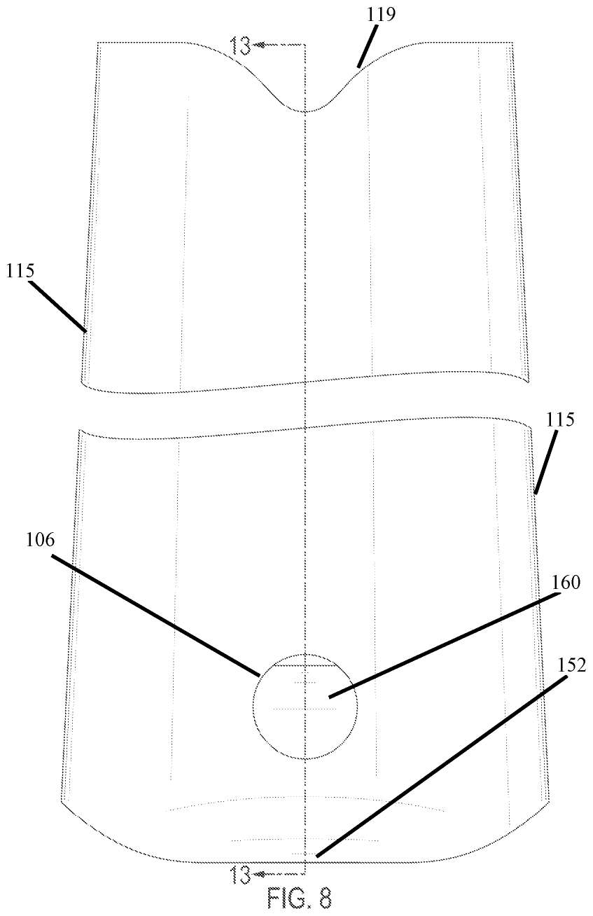

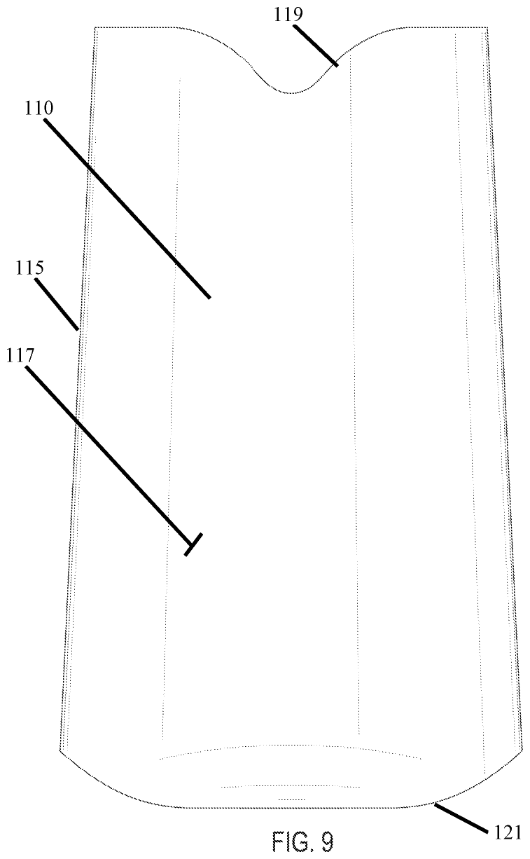

As discussed above, the distal ends of the front and back panel can be held together by the tab fixation section 162. Thus, the compression on the sealed inhaler can be greater at the distal end because the front and back panels are inhibited or prevented from bending as far apart as the proximal end, as shown, for example, in FIGS. 7, 10, and 13. This can create additional friction or stiction against the sealed inhaler. The tab can also be pressed against the sealed inhaler by the back panel, which can provide additional compression force on the sealed inhaler. The combined compression from the front and back panels and the tab guide end can beneficially prevent or reduce the likelihood of the inhaler from falling out of the transport wallet regardless of how it is oriented but does not affect the removal of the inhaler when needed.

As mentioned above, a transport wallet 100 of the subject invention can be formed from a flat sheet of material, known in the art as folding paperboard. The folding paperboard can be cut, such as, for example, die cut, to create a “folding cartonboard” 180 that can be folded into a medication transport wallet. The folding cartonboard can be cut with an outline, pattern, or shape 182 that resembles the profile of the medication transport wallet. The folding cartonboard can have strategically placed score lines 190 that facilitate folding to form the medication transport wallet. In addition to the tab 150, discussed above, the back panel 110 can further have a flap 195 extending from a lateral edge 115 opposite to the lateral edge shared with the front panel 105, an example, of which, is shown in FIGS. 14 and 15. When folded along a score line (F3), the front and back panels can be side by side with facing interior sides 116. The flap can also be folded along another score line 190 (F2) to position the flap adjacent to the front panel 105, to which it can be attached. In one embodiment, the flap has an attachment area 197 that can be attached to the front panel. In a further embodiment, the attachment area can attach the flap to the interior side 116 of the front panel 105 to form the lateral side 115 and enclose the interior space 120 of the transport wallet. The attachment area can be attached to the interior side 116 of the front panel 105 by any of a variety of devices, materials, and techniques known to those with skill in the art, including, but not limited to, adhesives, adhesive strips, hook-and-loop material, crimping, heat-sealing, and snaps. In a specific embodiment, as shown, for example, in FIG. 14, the attachment area is an adhesive strip arranged at or near to the score line 190 (see F2).

It can be beneficial if the dimensions of a transport wallet are such that it can receive an inhaler 16 within a protective cover 14, but still be small enough to be carried on or with a person, for example, in a pocket or purse. In one embodiment, a longitudinal length 124 of the transport wallet, which is the greatest distance between the proximal end 5 of a front or back panel and the distal end 10 where the panels are adjacent and attached with the tab 150, as shown in FIGS. 4, is between about 4.5″, 4.75″, 5.0″, 5.25″, 5.5″, 5.75″, 6.0″, 6.5″, 7.0″, 7.25″, 7.5″ and/or a longitudinal length between any two of the list values. In an alternative embodiment, the longitudinal length of the transport wallet is between about 11 cm, 12 cm, 13 cm, 14, cm, 15 cm, 16 cm, 17 cm, 18 cm, 19 cm, 20 cm and/or a longitudinal length between any two of the listed values. In a select embodiment, a transport wallet has a longitudinal length of about 6.37″ or, alternatively, a longitudinal length of about 16.18 cm. In a further embodiment, a width 113 of a transport wallet, which is the distance between the lateral edges when the transport wallet is flat, as shown in FIG. 3, is between about 2.5″, 3.0″, 3.5″, 3.75″, 4.0″, 4.25″, 4.5″, and/or a width between any two of the listed values. In an alternative embodiment, the width of a transport wallet is between about 6 cm, 7 cm, 8 cm, 9 cm, 10 cm, 11 cm, 12 cm, and/or a width between any two of the listed values. In a select embodiment, a transport wallet has width of about 3.74″ or, alternatively, a width of about 9.5 cm.

In some embodiments, a transport wallet 150 can be part of a kit 200. The kit can include an outer packaging 12 that contains one or more transport wallets and one or more inhalers 16 that are individually sealed within protective covers 14. In one embodiment, the kit can comprise an inhaler such as a disposable inhaler for treating seizures, such as, for example, epilepsy-related seizures, in a patient in need thereof. FIG. 17 shows a non-limiting example of a kit 200, configured for treating seizures. In a specific embodiment, the kit comprises an inhaler 16 that contains and/or dispenses an inhalable antiseizure medication. In a still more specific embodiment, the inhaler comprises and/or dispenses a benzodiazepine. In one embodiment, the benzodiazepine is at least one of midazolam and alprazolam. In a preferred embodiment, the benzodiazepine is alprazolam. U.S. Pat. No. 11,241,383, which is hereby incorporated by reference in its entirety, discloses a disposable inhaler for pulmonary self-administration of a condensation aerosolized benzodiazepine, specifically alprazolam.

More specifically, U.S. Pat. No. 11,241,383 discloses a breath-activated device that can be used to self-deliver an effective amount of alprazolam as an anti-seizure medication. Dosage delivery of a therapeutic amount of alprazolam can range from 0.125 mg to 4 mg. In some embodiments, the therapeutic dose is in the range of about 0.5 mg to about 4 mg, preferably about 1 mg to about 2 mg per dose, as needed to treat seizures. As aerosols, a dose of about 0.5 mg to about 4 mg, preferably about 0.5 mg to about 2 mg, even more preferably about 1 mg to about 2 mg alprazolam are generally provided per inspiration for the treatment of seizures.

The inhaler can be sealed within a protective cover, which can be at least partially made of aluminum. When removed from the protective cover, the patient need only take a single inspiration through the inhaler mouthpiece, as taught by the '383 patent, without any other coordination, action, or step and with a high reliability in delivery.

In one embodiment, a kit 200, configured for treating seizures, in a patient in need thereof, can comprise an outer package 12 that contains an inhaler 16, as taught by the '383 patent, along with a transport wallet 100, according to embodiments of the subject invention. In a further embodiment, the inhaler administers an anti-seizure dose of about 1 mg to about 2 mg of aerosolized alprazolam to a patient in need of treatment. As mentioned above, FIG. 17 shows a non-limiting example of a kit configured for treating seizures, in patient in need thereof. The kit can also comprise one or more of printed material, one or more additional transport wallets, and other accessories known to those with skill in the art.

To use the kit 200, the outer packaging 12 can be opened and the inhaler 16, sealed within the protective cover 14, can be removed, with the transport wallet. The inhaler in the protective cover can be inserted into the transport wallet 100 through the access 118, which can be opened by squeezing or pressing together the lateral edges 115 of the transport wallet, causing the front and back panels to bend in opposite directions. In one embodiment, the guide section 160 of the tab 150 can be pushed and held downwards or towards the back panel through the aperture 160, such that the fixations section 158 and the guide section 160 form a “V” shape as shown, for example in FIG. 13. The sealed inhaler can be simultaneously inserted through the access 118, such that the packaged inhaler is against the guide section. When the lateral edges are released, the front and back panels converge or partially unbend to press against the protective sealed cover of the inhaler and the back panel 110 further presses or pushes the guide section against the inhaler in the protective cover 14, to further secure or hold the packaged inhaler within the travel wallet 100. In an alternative embodiment, as the lateral edges are squeezed or pressed together, the guide section of the tab can be bent inward, towards the back panel 110, through the aperture 106 in the front panel 105. This can position the guide section on the interior space 120 of the travel wallet, without being held, for receiving the packaged inhaler. With this alternative embodiment, the lateral edges can also be released to create the same advantageous effect described above. FIG. 16 illustrates an example procedure, with the larger arrows indicating where to press or squeeze the transport wallet to open the access 118 and where to hold down or bend the guide section 160 of the tab 150. The smaller arrows indicate insertion of the medical device 16, shown here in a protective cover 14, through the access. A further benefit to the transport wallet is that, when the inhaler is removed from the protective cover, such as when administering the anti-seizure medication, the unpackaged inhaler can be inserted back into the transport wallet in the same manner as the packaged inhaler was inserted into the transport wallet. The unpackaged inhaler can also be securely held in the interior space 120. Thus, the transport wallet can be used to store both the unused and used inhaler or either a wrapped or unwrapped inhaler.

As discussed above, when the lateral edges 115 are released, the front 105 and back 110 panels come together. This can allow the front and back panels, as well as the tab 150 on the interior 120 of the transport wallet to press against and hold the inhaler, packaged or unpackaged, arranged within the transport wallet. This can also maintain the access in a partially open position. Further, the back panel can press against the sealed inhaler and simultaneously press the guide section 160 against the sealed inhaler, for increased compression at the distal end thereof. Advantageously, the tab assists the transport wallet in holding the inhaler, or other personally carried medical device, arranged within the interior space 120, regardless of the orientation of the transport wallet. The transport wallet can also have an appearance that camouflages, conceals, or at least partially hides the protective cover 14 (so as to be more discreet). The smaller profile transport wallet can also be more convenient to carry, can protect the sealed cover of the inhaler from damage, and can reduce the time required to prepare the inhaler for use.

In a specific embodiment, a transport wallet containing a packaged inhaler for administering an amount of alprazolam to a patient in need of treatment is utilized at the first indication of the onset of a partial or generalized epileptic seizure. More specifically, an amount of alprazolam can be administered to stop or reduce the severity or duration of several forms of seizures. This includes but is not limited to seizures lasting longer than 2, 3, or 5 minutes such as prolonged focal seizures or prolonged generalized seizures including, but not limited to, those generalized seizures starting with a flurry of absence seizures or myoclonic seizures, seizures associated with juvenile myoclonic epilepsy, acute repetitive seizures (seizure clusters), and/or seizures wherein the patient has a history of seizures manifesting over a period of several minutes or. The alprazolam can be administered during the early phase of a seizure such as in the aura phase, in particular if the patient has a record of seizure clusters or prolonged seizures with a tendency to develop into generalized tonic-clonic seizures or even status epilepticus.

When the inhaler is needed, the sealed, packaged inhaler can be removed by pulling it from the access of the transport wallet, with no additional action or manipulation of the transport wallet. Advantageously, the profile of the proximal edges 119 of the front and back panels, around the access 118, as well as the partially open access can be convenient for grasping the protective cover 14 and removing the sealed inhaler 16 from the interior space 120 of the transport wallet 100. This can also expedite preparation of the inhaler for use because there is no need to also open an outer packaging 12. The aperture 106 can also be, but is not required to be, utilized to contact and push or move the sealed inhaler towards the access between the proximal edges to further facilitate rapid removal from the transport wallet. Once removed from the transport wallet, the protective cover can be opened to access the inhaler, which can be activated for use. In a specific embodiment, the inhaler is activated by a single inhalation on the mouthpiece.

With respect to these various aspects and embodiments, which have been described herein, the present disclosure contemplates inter alia:
1. A medication transport wallet, configured to contain a sealed inhaler for carrying on a person, comprising:
   a front panel comprising an interior surface and exterior surface,
   a back panel comprising an interior surface and exterior surface, wherein the front panel and the back panel are fixedly attached along lateral edges, such that there is an interior space between the front panel and the back panel, for receiving the sealed inhaler therein;
   a tab comprising a fixation section and a guide section, wherein the tab is fixedly attached to a distal edge of the back panel and extends into the interior space; and
   a fixation area on a back side of the fixation section that attaches the fixation section to the interior surface of the front panel, at least a portion of the aperture is covered by the guide section.
2. A medication transport wallet according to embodiment 1, further comprising an aperture between the interior surface and the exterior surface of the front panel, wherein the guide section is contacted through the aperture.
3. A medication transport wallet according to embodiment 1 or 2, further comprising a section fold line between the fixation section and the guide section.
4. A medication transport wallet according to any one of embodiments 1, 2, or 3, wherein the front panel and the back panel comprise a distal end width and the fixation section comprises a width that covers at least about 10%, 15%, 20%, 25%, 30%, 35%, 40%, 45%, 50%, 55%, 60%, 65%, 70%, or 75% of the distal end width, such that there is at least one slit between a distal end of the front panel and a distal end of the back panel.
5. A medication transport wallet of according to any one of embodiments 1, 2, 3, or 4, further comprising an access at the proximal end that opens when the lateral edges are pressed together.
6. A medication transport wallet of according to any one of embodiments 1 to 5, wherein the front panel comprises a proximal edge and the back panel comprises a proximal edge, wherein the proximal edges are congruent.
7. A medication transport wallet of according to any one of embodiments 1 to 6, wherein the front panel has a proximal edge and the back panel has a proximal edge and at least a portion of one of the proximal edges is more distal than the other proximal edge.
8. A medication transport wallet according to any one of embodiments 1 to 7, wherein the front and back panel comprise a paperboard material.
9. A medication transport wallet according to any one of embodiments 1 to 8, wherein the paperboard material has a grammage of between approximately 255 g/m² and approximately 280 g/m².
10. A medication transport wallet according to any one of embodiments 1 to 9, wherein the paperboard material has a grammage of approximately 270 g/m².
11. A medication transport wallet according to any one of embodiments 1 to 10, wherein the paperboard material has a thickness of between approximately 400 μm and approximately 450 μm.
12. A medication transport wallet according to any one of embodiments 1 to 11, wherein the paperboard material has a thickness of approximately 425 μm.
13. A medication transport wallet according to any one of embodiments 1 to 12, wherein said wallet comprises a thickness between the exterior side of the front panel and the exterior side of the back panel of about 0.4 mm, 0.5 mm, 0.6 mm, 0.7 mm, 0.8 mm, 0.9 mm, 1.0 m, 1.1 mm, 1.2 mm, 1.3 mm, 1.4 mm or a thickness between any two of the listed values.
14. A medication transport wallet according to any one of embodiments 1 to 13, wherein said medication transport wallet is constructed from a folding cartonboard.
15. A medication transport wallet according to any one of embodiments 1 to 14, wherein the folding cartonboard comprises a flap that attaches to the interior surface of the front panel.
16. A kit for treatment of an acute medical condition, the kit comprising:
an inhaler within a protective cover for the treatment of the acute medical condition,
wherein the inhaler preferably comprises at least one medication for treating said acute medical condition;
a medication transport wallet comprising,
   a front panel comprising an interior surface and an exterior surface; and
   a back panel comprising an interior surface and an exterior surface, wherein the front panel and the back panel are attached along lateral edges, such that there is an interior space between the interior surface of the front panel and the interior surface of the back panel, in which the inhaler can be placed or contained.
17. A kit according to embodiment 16, wherein the medication transport wallet further comprises:
   a tab comprising a fixation section and a guide section with a section fold line therebetween, wherein the tab extends into the interior and the fixation section is attached to the interior surface of the front panel.
18. A kit according to embodiments 16 or 17, wherein the inhaler is sealed within a protective cover, wherein the inhaler in the protective cover is retained in the interior between the interior surface of the front panel and the guide section of the tab.
19. A kit according to any one of embodiments 16 to 18, wherein the front panel further comprises an aperture.
20. A kit according to any one of embodiments 16 to 19, wherein the aperture allows contact with the guide section.
21. A kit according to any one of embodiments 16 to 20, wherein: a) the inhaler delivers the at least one medication as a condensation aerosol; or b) comprises an anti-seizure medication.
22. A kit according to any one of embodiments 16 to 21, wherein the inhaler is breath activated.
23. A kit according to any one of embodiments 16 to 22, wherein the acute medical condition is a seizure, and the inhaler comprises an anti-seizure medication.
24. A kit according to embodiment 21 or 23, wherein the anti-seizure medication comprises a benzodiazepine.
25. A kit according to any one of embodiments 16 to 24, wherein the inhaler comprises one of midazolam and alprazolam, preferably for use as an antiseizure medication.
26. A kit according to any one of embodiments 16 to 25, wherein the inhaler is configured to deliver between about 1 mg and about 2 mg of alprazolam.
27. A kit according to any one of embodiments 16 to 26, wherein the inhaler is configured to deliver about 2 mg of alprazolam.
28. A kit according to any one of embodiments 23 to 27, wherein the seizure is prolonged.
29. A kit according to any one of embodiments 23 to 28, wherein the seizure is in an aura phase, is a generalized seizure starting with absence seizures, is a generalized seizure starting with myoclonic seizures, or is part of a seizure cluster (acute repetitive seizures) and wherein the inhaler is configured to deliver the antiseizure medication to the deep lung, thereby stopping the ongoing seizure.
30. A kit according to any one of embodiments 16 to 29, further comprising an outer packaging that contains the at least one medication transport wallet and the at least one sealed inhaler.
31. A method for protecting a sealed inhaler, the method comprising:
obtaining a medication transport wallet comprising,
   a front panel comprising an interior surface and an exterior surface,
   a back panel comprising an interior surface and an exterior surface, wherein the front panel and the back panel are attached along lateral edges, such that there is an interior space between the interior surface of the front panel and the interior surface of the back panel,
   an access between the front panel and the back panel through which the sealed inhaler is inserted into the interior space;;
pressing the lateral edges to bend the front panel and the back panel in opposite directions thereby opening the access to the interior space;
inserting the sealed inhaler through the access to the interior space of the medication transport wallet; and
releasing the lateral edges, whereby the front panel at least partially unbends to press against the sealed inhaler and the back panel at least partially unbends to simultaneously press against the sealed inhaler.
32. A method according to embodiment 31, wherein the medication transport wallet further comprises:
   a tab comprising a fixation section and a guide section, wherein the fixation section is fixedly attached to a distal edge of the back panel and extends into the interior space and the guide section is pressed against the medication transport wallet by the back panel;
   a fixation area on a back side of the fixation section that attaches the fixation section to the interior surface of the front panel,
   wherein the method further comprises, inserting the inhaler into the interior space between the interior side of the front panel and the guide section, such that the back panel pushes the guide section against the inhaler.
33. A method according to embodiment 31 or 32, further comprising an aperture between the external surface and the internal surface of the front panel, wherein aperture provides contact with the guide section, and the method further comprises contacting the guide section through the aperture to move the guide section toward the back panel prior to inserting the inhaler, such that the back panel presses the guide section against the inhaler.
34. A method according to any one of embodiments 31, 32, or 33, wherein the medication transport wallet further comprises a section fold line between the fixation section and the guide section of the tab, such that, when the guide section is moved, the tab folds along the section fold line.
35. A method for using an inhaler, the method comprising:
obtaining an inhaler that is sealed in a protective cover and arranged within a medication transport wallet, the medication transport wallet comprising,
   a front panel comprising an interior surface and an exterior surface with an aperture therethrough,
   a back panel comprising an interior surface and an exterior surface, wherein the front panel and the back panel are attached along lateral edges, such that there is an interior space between the interior surface of the front panel and the interior surface of the back panel,
   an access to the interior space between the front panel and the back panel; grasping the sealed inhaler between the front panel and the back panel; and
pulling the sealed inhaler from the interior space through the access of the medication transport wallet.
36. A method according to embodiment 35, further comprising:
contacting the inhaler sealed in the protective cover through the aperture; and
pushing the inhaler sealed in the protective cover toward the access.
37. A method according to embodiment 35 or 36, further comprising removing the inhaler from the protective cover.
38. A method according to any one of embodiments 35 to 37, wherein the inhaler comprises an anti-seizure medication.
39. A method according to any one of embodiments 35 to 38, wherein the inhaler delivers the anti-seizure medication as a condensation aerosol.
40. A method according to any one of embodiments 35 to 39, wherein the method further comprises activating the inhaler by a breath.
41. A method for stopping a seizure by the pulmonary administration of an anti-seizure medication, to a patient in need thereof, the method comprising:
   (a) obtaining an inhaler comprising an anti-seizure medication and being disposed within a medication transport wallet, said wallet comprising:
      a front panel comprising an interior surface and exterior surface with an aperture therethrough,
      a back panel comprising an interior surface and an exterior surface, wherein the front panel and the back panel are attached along lateral edges, such that there is an interior space between the front panel and the back panel, for receiving the inhaler;
   (b) removing the inhaler from the interior space;
   and
   (c) administering the antiseizure medication to the patient.
42. The method according to embodiment 41, wherein the inhaler is sealed in a protective cover when placed in the medication transport wallet and the method further comprises (b1) opening the protective cover after the inhaler is removed from the interior space of the medication transportation wallet and (b2) removing the inhaler from the opened cover.
43. The method according to embodiment 41 or 42, wherein the anti-seizure medication is a benzodiazepine selected from midazolam and alprazolam.
44. The method according to any one of embodiments 41 to 43, wherein the inhaler delivers the anti-seizure medication as a condensation aerosol and the method further comprises activating the inhaler to administer the anti-seizure medication.
45. The method according to any one of embodiments 41 to 44, wherein the inhaler is breath-activated.
46. The method according to any one of embodiments 41 to 45, wherein the condensation aerosol is self-administered.
47. The method according to any one of embodiments 41 to 46, wherein the inhaler is configured to deliver about 1 to about 2 mg of alprazolam.
48. The method according to any one of embodiments 41 to 47, wherein the inhaler is configured to deliver about 2 mg of alprazolam.
49. The method according to any one of embodiments 41 to 48, wherein the seizure is an ongoing seizure.
50. The method according to any one of embodiments 41 to 49, wherein the ongoing seizure is selected from a partial onset seizure and a generalized seizure.
51. The method according to any one of embodiments 41 to 50, wherein the ongoing seizure is at least one of in the aura phase, a partial onset seizure, a generalized seizure starting with absence seizures, a myoclonic seizure, and a seizure cluster (acute repetitive seizures).
52. The method according to any one of embodiments 41 to 51, wherein the seizure is prolonged.
53. The medication transport wallet, kit or method of any one of the preceding embodiments wherein the inhaler is disposable.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

I claim:

1. A medication transport wallet, configured to contain a sealed inhaler for carrying on a person, comprising:
a front panel comprising an interior surface and exterior surface,
a back panel comprising an interior surface and exterior surface, wherein the front panel and the back panel are fixedly attached along lateral edges, such that there is an interior space between the front panel and the back panel, for receiving the sealed inhaler therein;
a tab comprising a fixation section and a guide section, wherein the tab is fixedly attached to a distal edge of the back panel and extends into the interior space; and
a fixation area on a back side of the fixation section that attaches the fixation section to the interior surface of the front panel, at least a portion of the aperture is covered by the guide section.

2. The medication transport wallet according to claim 1, further comprising an aperture between the interior surface and the exterior surface of the front panel, wherein the guide section is contacted through the aperture.

3. The medication transport wallet according to claim 1, further comprising a section fold line between the fixation section and the guide section.

4. The medication transport wallet according to claim 1, wherein the front panel and the back panel comprise a distal end width and the fixation section comprises a width that covers at least about 10%, 15%, 20%, 25%, 30%, 35%, 40%, 45%, 50%, 55%, 60%, 65%, 70%, or 75% of the distal end width, such that there is at least one slit between a distal end of the front panel and a distal end of the back panel.

5. The medication transport wallet according to claim 1, further comprising an access at the proximal end that opens when the lateral edges are pressed together.

6. The medication transport wallet according to claim 1, wherein the front panel comprises a proximal edge and the back panel comprises a proximal edge, wherein the proximal edges are congruent.

7. The medication transport wallet according to claim 1, wherein the front panel has a proximal edge and the back panel has a proximal edge and at least a portion of one of the proximal edges is more distal than the other proximal edge.

8. The medication transport wallet according to claim 5, wherein the front and back panel comprise a paperboard material.

9. The medication transport wallet according to claim 8, wherein the paperboard material has a grammage of between approximately 255 g/m² and approximately 280 g/m².

10. The medication transport wallet according to claim 9, wherein the paperboard material has a grammage of approximately 270 g/m².

11. The medication transport wallet according to claim 10, wherein the paperboard material has a thickness of between approximately 400 μm and approximately 450 μm.

12. The medication transport wallet according to claim 11, wherein the paperboard material has a thickness of approximately 425 μm.

13. The medication transport wallet according to claim 1, wherein said wallet comprises a thickness between the exterior side of the front panel and the exterior side of the back panel of about 0.4 mm, 0.5 mm, 0.6 mm, 0.7 mm, 0.8 mm, 0.9 mm, 1.0 m, 1.1 mm, 1.2 mm, 1.3 mm, 1.4 mm or a thickness between any two of the listed values.

14. The medication transport wallet according to claim 13, wherein said medication transport wallet is constructed from a folding cartonboard.

15. The medication transport wallet according to claim 14, wherein the folding cartonboard comprises a flap that attaches to the interior surface of the front panel.

16. A kit for treatment of an acute medical condition, the kit comprising:
an inhaler within a protective cover, wherein the inhaler comprises at least one medication for treating the acute medical condition;
a medication transport wallet comprising,
a front panel comprising an interior surface and an exterior surface; and
a back panel comprising an interior surface and an exterior surface, wherein the front panel and the back panel are attached along lateral edges, such that there is an interior space between the interior surface of the front panel and the interior surface of the back panel, in which the inhaler is placed.

17. A method for protecting a sealed inhaler, the method comprising:
obtaining a medication transport wallet comprising,
a front panel comprising an interior surface and an exterior surface,
a back panel comprising an interior surface and an exterior surface, wherein the front panel and the back panel are attached along lateral edges, such that there is an interior space between the interior surface of the front panel and the interior surface of the back panel,
an access between the front panel and the back panel through which the sealed inhaler is inserted into the interior space;
pressing the lateral edges to bend the front panel and the back panel in opposite directions thereby opening the access to the interior space;
inserting the sealed inhaler through the access to the interior space of the medication transport wallet; and
releasing the lateral edges, whereby the front panel at least partially unbends to press against the sealed inhaler and the back panel at least partially unbends to simultaneously press against the sealed inhaler.

18. A method for using an inhaler, the method comprising:
obtaining an inhaler that is sealed in a protective cover and arranged within a medication transport wallet, the medication transport wallet comprising,
a front panel comprising an interior surface and an exterior surface with an aperture therethrough,
a back panel comprising an interior surface and an exterior surface, wherein the front panel and the back panel are attached along lateral edges, such that there is an interior space between the interior surface of the front panel and the interior surface of the back panel,
an access to the interior space between the front panel and the back panel;
grasping the sealed inhaler between the front panel and the back panel; and
pulling the sealed inhaler from the interior space through the access of the medication transport wallet.

19. A method for stopping an ongoing seizure by the pulmonary administration of an anti-seizure medication, to a patient in need thereof, the method comprising:
(a) obtaining an inhaler comprising an anti-seizure medication and that is placed within a medication transport wallet, wherein the medication transport wallet comprises:
a front panel comprising an interior surface and an exterior surface with an aperture therethrough,
a back panel comprising an interior surface and an exterior surface, wherein the front panel and the back panel are attached along lateral edges, such that there is an interior space between the front panel and the back panel, for placing the inhaler;
(b) removing the inhaler from the interior space; and
(c) administering the antiseizure medication to the patient.
